(19) United States
(12) Patent Application Publication
(10) Pub. No.: US 2024/0082507 A1
(43) Pub. Date: Mar. 14, 2024

(54) CASSETTE FOR AN AUTOINJECTOR AND RELATED METHODS

(71) Applicant: AMGEN INC., Thousand Oaks, CA (US)

(72) Inventors: Samin Akbari, Cambridge, MA (US); Azita Rahbari, Westlake Village, CA (US); Sunitha Dasoju, Ventura, CA (US); Andrew Coles, Thousand Oaks, CA (US); Jerome Olivas, Thousand Oaks, CA (US); Michele Macchi, Milan (IT); Desheng Yin, Thousand Oaks, CA (US); Harpreet Sachar, Newbury Park, CA (US); Alireza Ashani, Woodland Hills, CA (US); Angelo Tosarini, Milano (IT); Antonio Antonini, Milano (IT)

(21) Appl. No.: 17/767,462

(22) PCT Filed: Oct. 7, 2020
(86) PCT No.: PCT/US20/54536

§ 371 Date: Apr. 8, 2022

Related U.S. Application Data
(60) Provisional application No. 62/912,540, filed on Oct. 8, 2019.

(51) Int. Cl. A61M 5/32

(52) U.S. Cl. CPC A61M 5/3257; A61M 5/3213

(57) ABSTRACT

A cassette for a drug delivery device is described that includes a sleeve, a syringe having a barrel disposed in the sleeve, and a plunger-stopper slidably disposed within the barrel. The cassette further includes a spacer that is configured to be coupled to the sleeve. The cassette can form a part of an apparatus for injection of a therapeutic product along with a drug delivery device.

### CROSS-REFERENCE TO RELATED APPLICATION

This application claims the priority benefit of U.S. Provisional Application No. 62/912,540, filed Oct. 8, 2019, which is hereby incorporated by reference herein in its entirety.

### FIELD OF DISCLOSURE

The present disclosure generally relates to drug delivery devices and, more particularly, to autoinjector devices.

### BACKGROUND

Pre-filled hypodermic syringes provide several advantages for the home-use market. These advantages include that pre-filled syringes may be prepared for each medicament with exactly the required dosage. Further, they are easily operated, by merely advancing the plunger-stopper of the syringe. Aside from the costs of the particular medication used, pre-filled syringes are also economically manufactured. Consequently, all these advantages make pre-filled syringes commercially appealing.

Nevertheless, pre-filled syringes also have a significant drawback in the marketplace. Specifically, many users are either frightened by an exposed needle or feel they are inherently incapable of performing an injection. Because of aversions to exposed needles, as well as health and safety issues that may be involved, various types of injectors and other devices have been developed for the specific purpose of concealing needles from the user and automating the injection task to assist the user in performing the injection. One such injector is a reusable autoinjector that receives cartridges having a pre-filled syringe therein. A user orients the autoinjector at a desired injection location, actuates a user input, and a drive or drives of the autoinjector moves the syringe to insert the needle to a subcutaneous location and extrudes a dose of a drug from the syringe with a plunger rod engaging and driving a plunger-stopper through a barrel of the syringe.

Different syringes having varying ranges of barrel diameters can used in the same autoinjector. The plunger-stoppers for such syringes have a similar range of diameters. The size and geometry of the plunger rod used for engaging the variety of plunger-stoppers, however, tends to remain static. A plunger rod suitable for a small diameter barrel and plunger-stopper may provide unsatisfactory operation when used in a larger diameter barrel with a larger plunger-stopper and vice versa.

### SUMMARY

In accordance with a first aspect, a cassette for a drug delivery device is described that includes a sleeve having a proximal end and a distal end having an opening, a syringe disposed in the sleeve, where the syringe includes a barrel having a distal opening coaxially aligned with the opening of the distal end of the sleeve, and a plunger-stopper slidably disposed within the barrel. The cassette further includes a spacer having a proximal end and a distal end, where the distal end is configured to be inserted into the opening to couple the spacer to the sleeve. The distal end of the spacer is adapted to be engaged by a plunger rod of a drive mechanism to uncouple the spacer from the sleeve and slide the spacer within the barrel to engage the plunger-stopper with the proximal end thereof.

According to some forms, the sleeve can include an annular wall extending around the opening and the distal end of the spacer can be configured to engage an interior surface of the annular wall.

According to some forms, the sleeve can include a lock cap that is configured to secure the syringe in the sleeve. In further forms, the opening of the sleeve can be defined by a portion of the lock cap extending over the distal opening of the barrel, such that the spacer is configured to couple to the lock cap. In some forms, the portion of the lock cap can be a generally planar body and the annular wall can be integral with the body. In these forms, the lock cap can further include a gasket that is configured to couple to the body with a main face of the gasket extending along an interior surface of the body, where the main face defines an opening configured to coaxially align with the opening of the body such that the distal end of the spacer is configured to be inserted through the opening of the gasket. In further forms, the spacer can include a neck portion disposed between and having a reduced diameter relative to the proximal and distal ends to define a space therebetween, and the opening of the gasket can have a diameter sized so that portions of the main face extend into the space between the proximal and distal ends of the spacer and/or the gasket can include one or more rims extending away from the main face, where the rims include lips configured to engage the body to couple the gasket thereto. In any of the above forms, the sleeve can include a cover that is configured to couple to the distal end thereof with the lock cap disposed proximal of the cover, where the cover includes an opening extending therethrough and an annular wall extending around the opening and extending in a proximal direction, such that the annular wall of the cover extends around the annular wall of the lock cap. In some forms, the lock cap can include a tubular member including the annular wall and a generally planar body having an annular configuration, where the tubular member is coupled to the body with the annular wall extending through the body. In some forms, the tubular member can include a flange extending along the body, where the body and tubular member are overmolded together with connection posts of the tubular member extending from the flange through openings in the body and/or the spacer can include a neck portion disposed between and having a reduced diameter relative to the proximal and distal ends to define a space therebetween and the tubular member can include one or more projections that extend radially inward from the annular wall, where the projections sized to at least partially extend into the space between the proximal and distal ends of the spacer.

The cassette according to any of the above forms can include one or more of the following aspects: the proximal end of the spacer can have a diameter approximately equal to a diameter of the plunger-stopper; the proximal end of the spacer can include one or more grooves extending along an outer surface thereof; the distal end of the spacer can include a plurality of ribs extending radially outwardly therefrom, where the plurality of ribs provide an outer diameter from the distal end to frictionally engage the lock cap; the spacer can have a cup-shaped configuration with a distal end wall and a cavity having an opening extending through the proximal end; a distal end surface of the spacer can be configured to be coplanar with a distal end surface of the lock cap with the spacer frictionally coupled thereto; the cassette can include an outer housing configured to movably receive the sleeve and syringe therein; or the cassette can include a therapeutic product in the syringe.

In accordance with a second aspect, an apparatus for injection of a therapeutic product is described that includes a drug delivery device comprising a drive and a plunger rod, and a cassette for use with the drug delivery device. The cassette can have any of the forms described above.

In accordance with a third aspect, a method for preparing a cassette for an autoinjector is described that includes disposing a plunger-stopper within a barrel of a syringe, disposing the syringe within a sleeve, inserting a distal end of a spacer into an opening of the sleeve to couple the spacer thereto, where the opening is aligned with a distal opening of the barrel of the syringe to coaxially align the spacer with the barrel of the syringe.

According to some forms, inserting the distal end of the spacer into the opening of the sleeve can include inserting the distal end of the spacer into an opening of a lock cap to couple the spacer thereto; and the method can further include coupling the lock cap to a distal end of the sleeve such that the spacer is coaxially aligned with the distal opening of the barrel of the syringe. In some forms, inserting the distal end of the spacer into the opening the lock cap can include inserting the distal end into a cavity defined by an annular wall of the lock cap, where the distal end is configured to frictionally engage an interior surface of the annular wall.

According to further forms, the method can include one or more of the following aspects, inserting the distal end of the spacer into the cavity defined by the annular wall further can include inserting the distal end of the spacer through an opening in a gasket or member coupled to a body of the lock cap such that a portion of the gasket or member extends into a space between the distal end of the spacer and a proximal end of the spacer; the method can include selecting the spacer based on a size of the plunger-stopper and a size of a proximal end of the spacer, the method can include selecting the sleeve based on a size of an interior bore defined by one or more interior walls of the sleeve and a diameter of the barrel of the syringe, or the method can include filling the syringe with a therapeutic product.

In accordance with a fourth aspect, a method of assembling a cassette for a drug delivery device is described that includes selecting a syringe having a barrel with an outer diameter, selecting a sleeve from first and second sleeves, the first and second sleeves having a common outer configuration, common outer dimensions, and internal bores defined by one or more walls having different diameters, the selection of the sleeve comprising selection of one of the first and second sleeves having an internal bore sized to support the barrel of the syringe, inserting the syringe into the sleeve, and inserting the syringe and sleeve into a housing, the housing configured to couple to the common outer configuration of the first and second sleeves.

According to some forms, the internal bores of the first and second sleeves can be defined by an array of radial ribs extending within an interior of the sleeve.

### BRIEF DESCRIPTION OF THE DRAWINGS

FIG. 1 is an elevational side view of an exemplary embodiment of an autoinjector apparatus including an autoinjector and a cassette.

FIG. 2 is an exploded perspective view of an exemplary embodiment of the cassette of FIG. 1 showing an outer housing, an inner sleeve, a syringe, a shield remover, a lock cop, and a cover.

FIG. 3 is a top down front perspective view of the cassette of FIG. 1.

FIG. 4 is a sectional side view of the cassette of FIG. 1.

FIG. 5 is a front perspective view of an example lock cap for a sleeve.

FIG. 6 is a rear perspective view of a portion of a sleeve with the lock cap of FIG. 5 and a syringe.

FIG. 7 is a side view of a portion of the sleeve with the lock cap of FIG. 6 and the syringe.

FIG. 8 is a front perspective view of a portion of a sleeve with a second example lock cap.

FIG. 9 is a perspective view of a first example spacer.

FIG. 10 is a perspective view of a second example spacer.

FIG. 11 is a bottom perspective view of a first example lock cap for a sleeve of a cassette having an annular wall to receive a portion of a spacer therein.

FIG. 12 is a top perspective view of the lock cap of FIG. 11.

FIG. 13 is a cross-sectional exploded view of a first example cassette showing a cover, a sleeve with the lock cap of FIG. 11, the spacer of FIG. 9 coupled to the sleeve, and a syringe.

FIG. 14 is a sectional perspective view of the cassette of FIG. 13.

FIG. 15 is a cross-sectional exploded view of a second example cassette showing a cover, a sleeve with the lock cap of FIG. 11, the spacer of FIG. 10 coupled to the sleeve, and a syringe.

FIG. 16 is a sectional perspective view of the cassette of FIG. 13.

FIG. 17 is a perspective view of a gasket for a lock cap.

FIG. 18 is a perspective view of a second example lock cap for a sleeve and the spacer of FIG. 10, the lock cap including the gasket of FIG. 17.

FIG. 19 is a cross-sectional view of the lock cap and spacer of FIG. 18.

FIG. 20 is a perspective view of a third example lock cap for a sleeve including a tubular member coupled to a body.

FIG. 21 is a cross-sectional view of the lock cap of FIG. 20.

FIG. 22 is a cross-sectional view of the lock cap of FIG. 20 including the spacer of FIG. 9.

FIG. 23 is a perspective view of a first example sleeve for a cassette.

FIG. 24 is a perspective view of a second example sleeve for a cassette.

FIG. 25 is a bottom up, front perspective view of the cassette of FIG. 1 showing a bottom surface with projections.

FIG. 26 is a bottom view of the cassette of FIG. 25 showing the projections and a latch mechanism.

FIG. 27 is a front elevational view of the autoinjector of FIG. 1.

FIG. 28 is an elevational view of the autoinjector FIG. 1.

FIG. 29 is a rear elevational view of the autoinjector of FIG. 1.

FIG. 30 is an elevational view of a second side of the autoinjector of FIG. 1.

FIG. 31 is an elevational view of a first end of the autoinjector of FIG. 1.

FIG. 32 is an elevational view of a second end of the autoinjector of FIG. 1.

FIG. 33 is a sectional side view of the autoinjector and cassette of FIG. 1.

FIG. 34 is a top down perspective side view of an example motorized insertion drive 330 for the autoinjector of FIG. 1.

FIG. 35 is a bottom up perspective view of the motorized insertion drive of FIG. 34.

FIG. 36 is an exploded perspective side view of a plunger rod, a lead screw, and a nut of the motorized extrusion drive for the autoinjector of FIG. 1.

FIG. 37 is an assembled perspective side view of the plunger rod, the lead screw, and the nut of FIG. 36.

FIG. 38 is a perspective view of a portion of the motorized extrusion drive of FIGS. 34-37.

### DETAILED DESCRIPTION

A cassette for a drug delivery device, an apparatus for the injection of a therapeutic product, and related methods are described herein that utilize a spacer to provide an intermediary member between a plunger-stopper of a syringe of the cassette and a plunger rod of the drug delivery device. The spacer can couple with a sleeve of the cassette in a press-fit engagement, which allows the spacer to be reliably uncoupled by the plunger rod during an extrusion process. Further, a proximal end of the spacer can be specifically tailored for a particular plunger-stopper and barrel size to ensure proper engagement and seating between the components. The spacers can include venting features so that air is not trapped between the spacer and the plunger-stopper when the two objects are moved relative to one another within the barrel. In a drug extrusion operation, the spacer is spaced from the plunger-stopper and engaged by a plunger rod to slide within the barrel and engage the plunger-stopper.

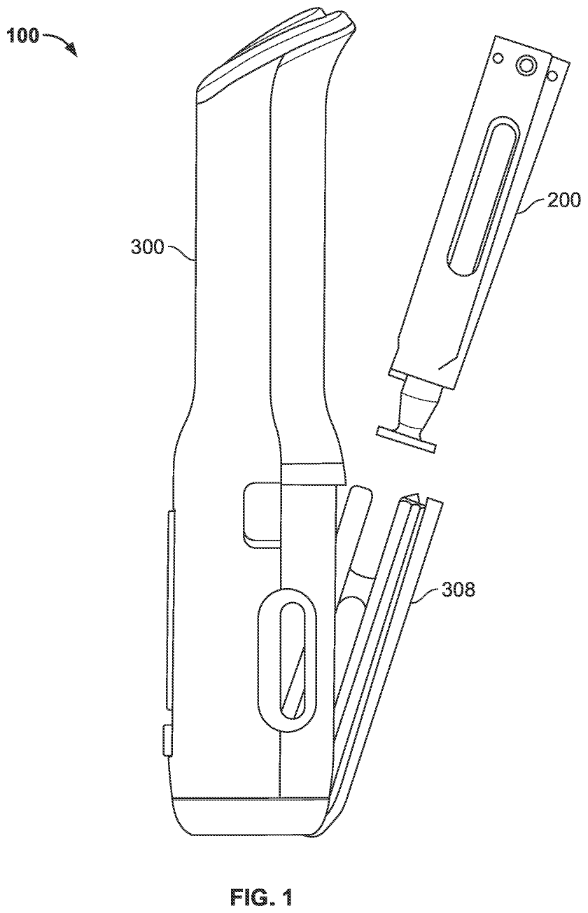

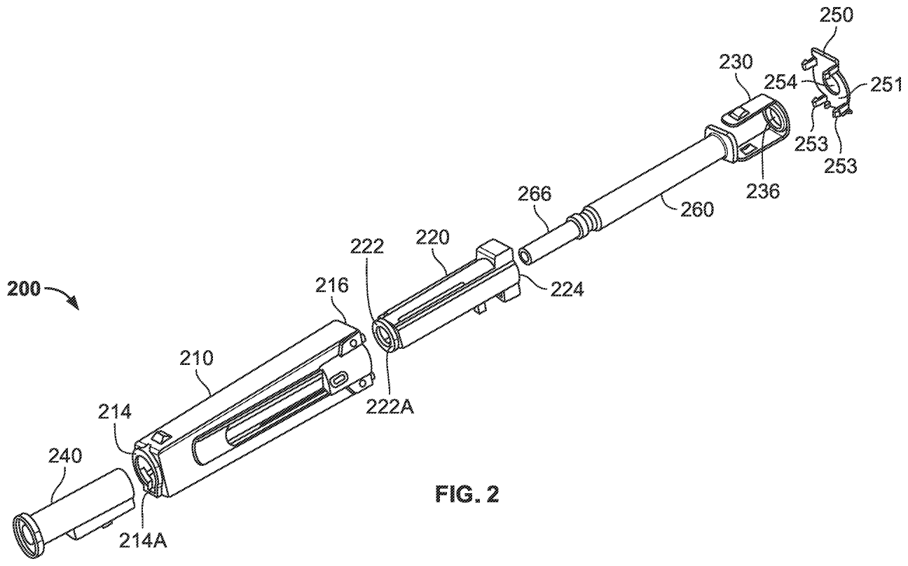

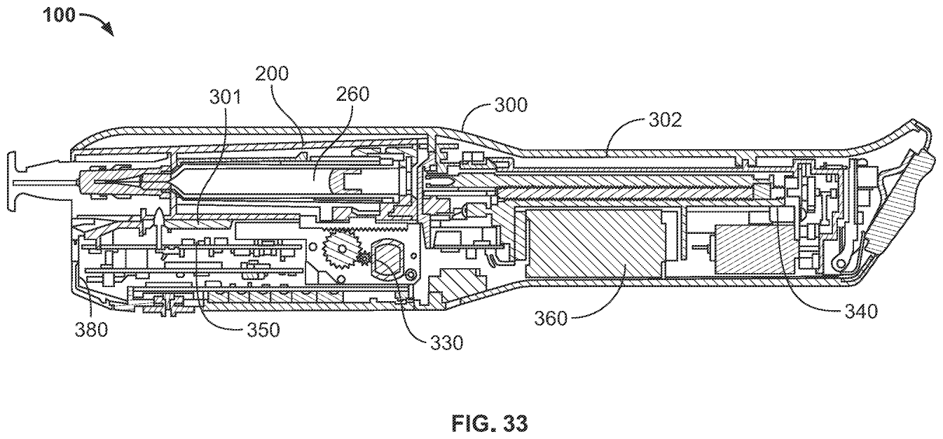

FIG. 1 illustrates an elevational view of an exemplary embodiment of an autoinjector apparatus 100 according to the present disclosure. The autoinjector apparatus 100 comprises an autoinjector 300 and a cassette 200. The autoinjector 300 may comprise a cassette door 308, which in an open position, (as shown) allows insertion therein of the cassette 200, and which in a closed position (e.g., FIG. 28), aligns the cassette 200 with insertion and extrusion drives 330 and 340, respectively (FIG. 33) of the autoinjector 300. The autoinjector 300 may be constructed and adapted for hand-held operation and be reusable. The cassette 200 may be constructed and adapted to house and protect a syringe 260 (e.g., FIG. 2), which may be prefilled with a predetermined dose of a pharmaceutical product. The cassette 200 facilitates and enables easy use of the syringe with the autoinjector 300 and helps prevent needle sticks before and after use. Moreover, the cassette 200 may be constructed and adapted for single, disposable use.

FIG. 2 illustrates an exploded perspective view of an exemplary embodiment of the cassette 200, according to the present disclosure. The cassette 200 may comprise an outer housing 210, an inner sleeve 220 slidably moveable within the outer housing 210, a syringe 260 disposed within or held by the inner sleeve 220, and a shield remover 240 for removing a protective needle shield 266 of the syringe 260. The outer housing 210 may comprise a proximal end wall 214 and an open distal end 216. The proximal end wall 214 of the outer housing 210 may include an aperture 214A having a size and shape for receiving therethrough the shield remover 240. The inner sleeve 220 may comprise a proximal end wall 222 and an open distal end 224. The proximal end wall 222 of the inner sleeve 220 may include an aperture 222A having a size and shape for receiving therethrough the protective needle shield 266 of the syringe 260. The sleeve 220 may further comprise an end or lock cap 230 for closing the open distal end 224 of the inner sleeve 220 and securing or locking the syringe 260 within the inner sleeve 220. The cassette 200 may further comprise a cover 250 for closing the open distal end 216 of the outer housing 210. The cover 250 provides for tamper resistance by encasing the inner sleeve 220 and the syringe 260 containing a pharmaceutical product 267, within the outer housing 210 of the cassette 200, and also completes the cosmetic appearance of the cassette 200.

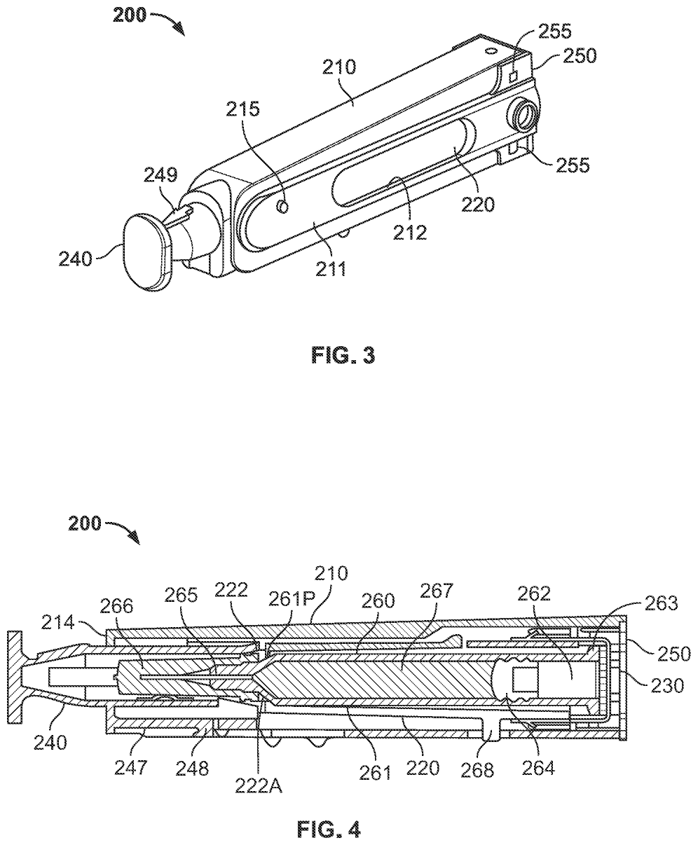

FIG. 3 illustrates a top down front perspective view of the cassette 200. The outer housing 210 of the cassette 200 may comprise an elongated opening or window 212 in each side wall 211 thereof. The windows 212 may be disposed opposite to and aligned with one another. Further, the inner sleeve 220 of the cassette 200 may be made from a transparent, rigid material, such as a clear polycarbonate. The windows 212 in the side walls 211 of the outer housing 210 in combination with the transparent inner sleeve 220, allow viewing of the syringe 260 housed within the inner sleeve 220 (FIG. 4). The wall portions of the inner sleeve 220 viewable through the windows 212 of the outer housing 210 may comprise fill volume indicia (not shown). The outer housing 210 of the cassette 200 may also include a pin 215 or any other suitable mechanical structure that prevents the cassette 200 from being inserted into the cassette door 308 in the wrong direction and/or orientation. An “arrow” icon may be provided on the shield remover 240 or the outer housing 210 (not shown) to indicate the proper direction and orientation of cassette insertion into the cassette door 308.

FIG. 4 illustrates a sectional side view of the cassette 200. As can be seen, the inner sleeve 220 may comprise an inner sleeve pin 268, which may be engaged by an insertion drive 330 of the autoinjector 300 (FIG. 33) during the operation thereof. When driven by the insertion drive 330, the pin 268 moves the inner sleeve 220 within the outer housing 210 of the cassette 200. The inner sleeve 220 may be sized and shaped to receive the syringe 260 therein.

Referring still to FIG. 4, the syringe 260 may comprise a barrel 261 that defines a fluid chamber 262. The fluid chamber 262 may be prefilled with a predetermined dose of a pharmaceutical product 267. The pharmaceutical product 267 may have a viscosity that depends on the temperature of the product 267. The syringe 260 may further comprise an injection needle 265 removably or fixedly disposed at a proximal end of the barrel 261, and an outwardly extending flange 263 disposed at a distal end of the barrel 261. The injection needle 265 may communicate with the fluid chamber 262 to allow dispensing of the predetermined dose of a pharmaceutical product 267 expelled from the fluid chamber 262 of the syringe barrel 261. The syringe 260 may further comprise a moveable plunger-stopper 264, disposed within the fluid chamber 262 of the barrel 260, for expelling the predetermined dose of the pharmaceutical product 267 from the chamber 261 so that it may be dispensed through the injection needle 265. The protective needle shield 266 mentioned earlier, covers the injection needle 265 and may be made of a non-rigid material. In one exemplary embodiment, the syringe 260 may comprise a standard 1-mL long glass syringe. The lock cap 230 closes the distal end 224 of the inner sleeve 220 and fixedly secures a proximal end 261P of the syringe barrel 261 against an inner edge surface formed at the junction of the interior surface of the proximal end wall 222 and the aperture 222A of the inner sleeve 220, so that the syringe 260 moves with the inner sleeve 220 as it travels within the outer housing 210, during the operation of the autoinjector 300.

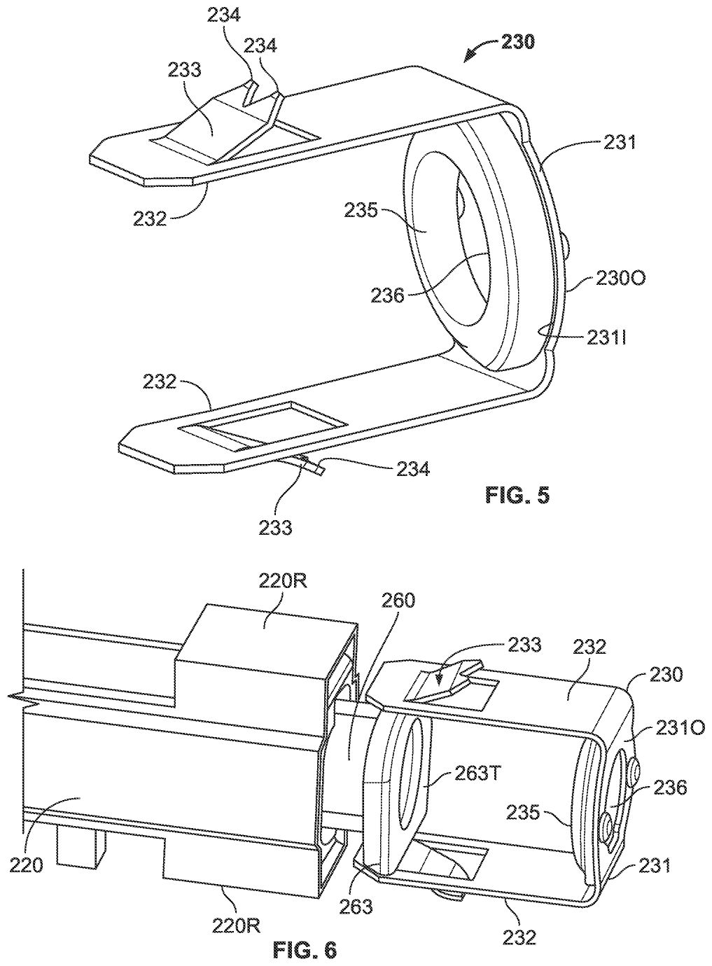

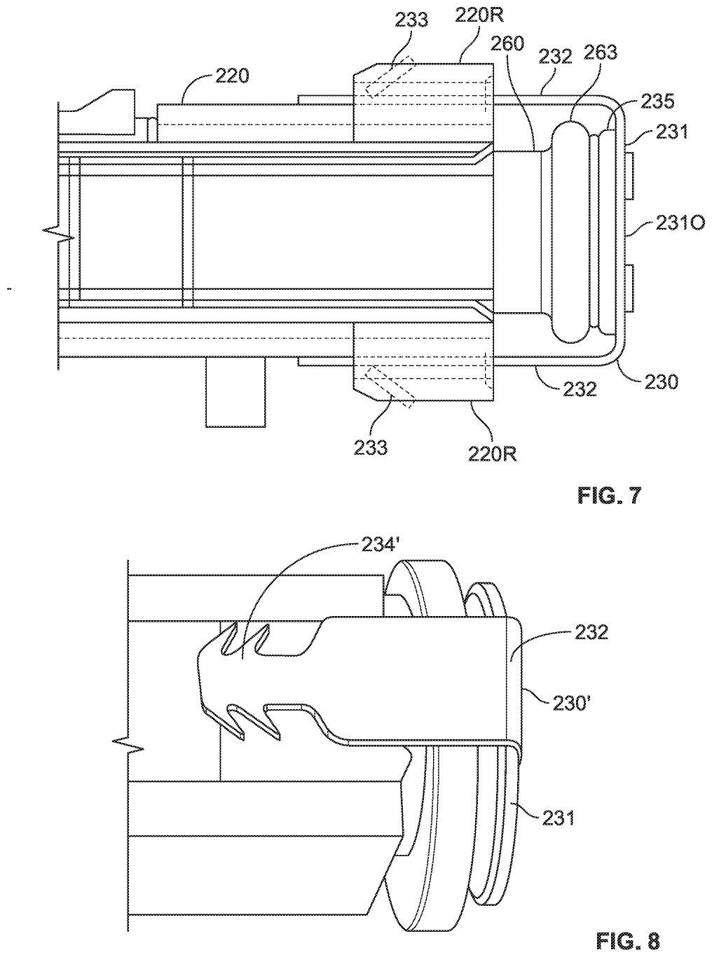

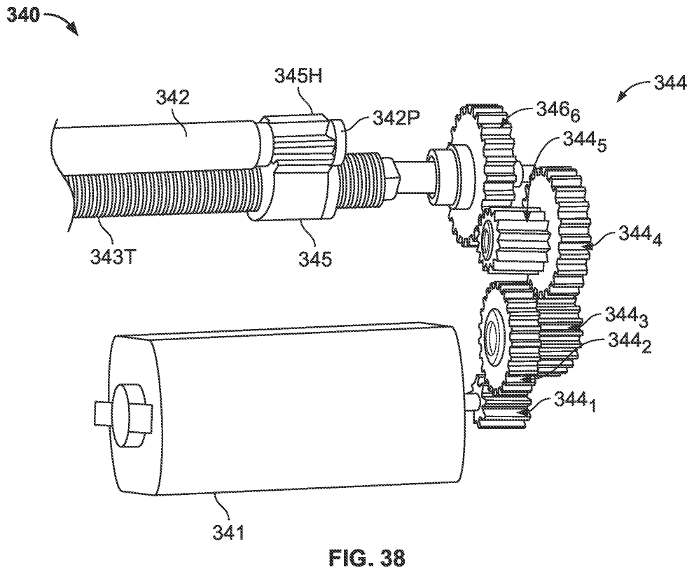

The lock cap 230, illustrated in FIGS. 5-7, locks the syringe 260 in the inner sleeve 220 with a predetermined force which may be set during assembly of the cassette 200. The lock cap 230 may comprise a generally flat, annular body 231 having outer and inner surfaces 2310 and 2311, and opposing arms 232 depending from the body 231, away from the inner surface 2311 thereof. Each of the arms 232 may comprise a cut-out member 233 with a barbed end 234. In some embodiments, the cut-out members 233 may be spring-like. The members 233 may extend outwardly from the arms 232 and toward the body 231. The body 231 can be made from a metal or rigid plastic material. A soft elastomeric ring-shape bumper 235 may be affixed to the inner surface 2311 of the body 231. The body 231 and bumper 235 may define an opening 236 which can be dimensioned to allow a plunger rod 342 actuated by a motorized extrusion drive 340 of the autoinjector 300 (FIG. 38), to pass through the lock cap 230 and engage and move the plunger-stopper 264 through the fluid chamber 262 of the syringe barrel 261 during the operation of the autoinjector 300. The lock cap 230 may be dimensioned to receive the flange 263 of the syringe 260 between the opposing arms 232 thereof, in a slip-fit manner with the bumper 235 engaging a top surface 263T of the flange 263 as illustrated in FIGS. 6 and 7. The arms 232 of the lock cap 230 may be inserted into opposing receiving receptacles 220R formed at a distal end of the inner sleeve 220 when the syringe 260 is assembled into the inner sleeve 220. The barbs 234 of the arms 232 grip the inner surfaces of the receiving receptacles 220R to lock the lock cap 230 into position, thereby lockingly holding the syringe 260 in the inner sleeve 220. The arms 232 of the lock cap 230 may be inserted into the receptacles 220R of the inner sleeve 220 a selected distance to limit the amount of force (to a predetermined value) applied to the syringe 260 during assembly into the cassette 200 and during usage.

FIG. 8 illustrates an alternate embodiment of the lock cap numbered 230′. The lock cap 230′ is similar to the lock cap 230 of FIGS. 5-7, but omits the cut-out members 233 and instead, provides a barb arrangement 234′ at the end of each arm 262.

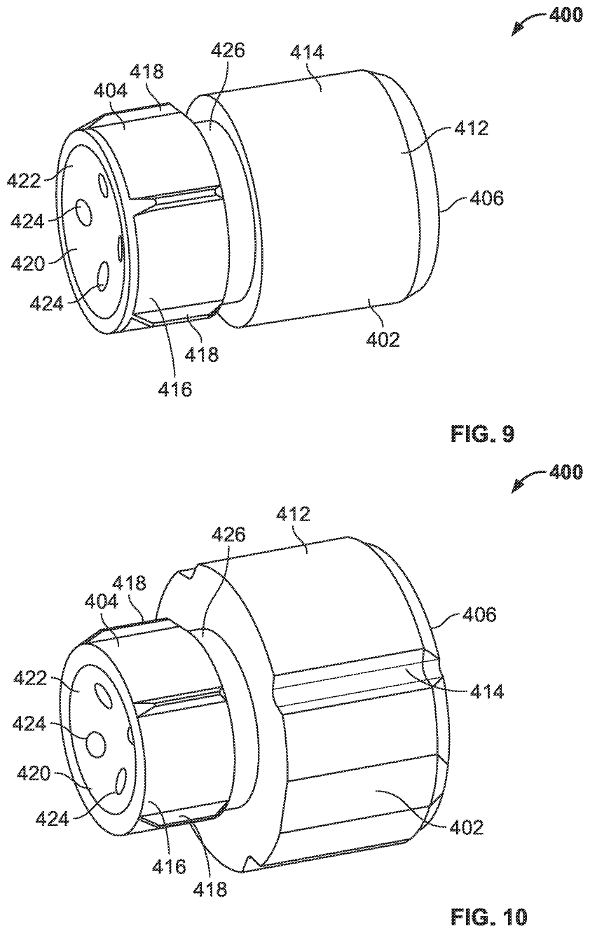

As shown in FIGS. 9-22, a spacer 400 can be utilized in the cassette 200 so that the injector 100, and the extrusion drive 340 and plunger rod 342 thereof, can be utilized with a variety of syringe 260 sizes while ensuring proper engagement and drug extrusion. The spacer 400 can function as an intermediate component between the plunger-stopper 264 of the syringe 260 and the plunger rod 342 with a proximal end 402 configured to engage the plunger-stopper 264 and a distal end 404 configured to engage the plunger rod 342. The spacer 400 acts as an adapter so that the size of the barrel 261 and plunger-stopper 264 can be scaled as desired, while the plunger rod 342 can have a reusable single size in the autoinjector 300. The spacer 400 can advantageously decrease the length of the plunger rod 342 stroke to deliver a full dose of the drug and, as such, will reduce the injection time and can reduce the total length of the autoinjector 300. Thus, as the cassette 200 is assembled, the spacer 400 can be selected from a plurality of available spacers, each having plunger-stopper engagement portions having different diameters and, if desired, other dimensions, to have a diameter that matches with the syringe barrel size and plunger-stopper size for any given application. Additionally, opposite ends of the various spacers 400 can have a uniform configuration, such that all of the spacers can couple to the same cassette component. The various configurations described herein can also avoid potential operational issues, such as misalignment of the syringe and plunger rod, binding of the plunger rod to the plunger-stopper, and so forth.

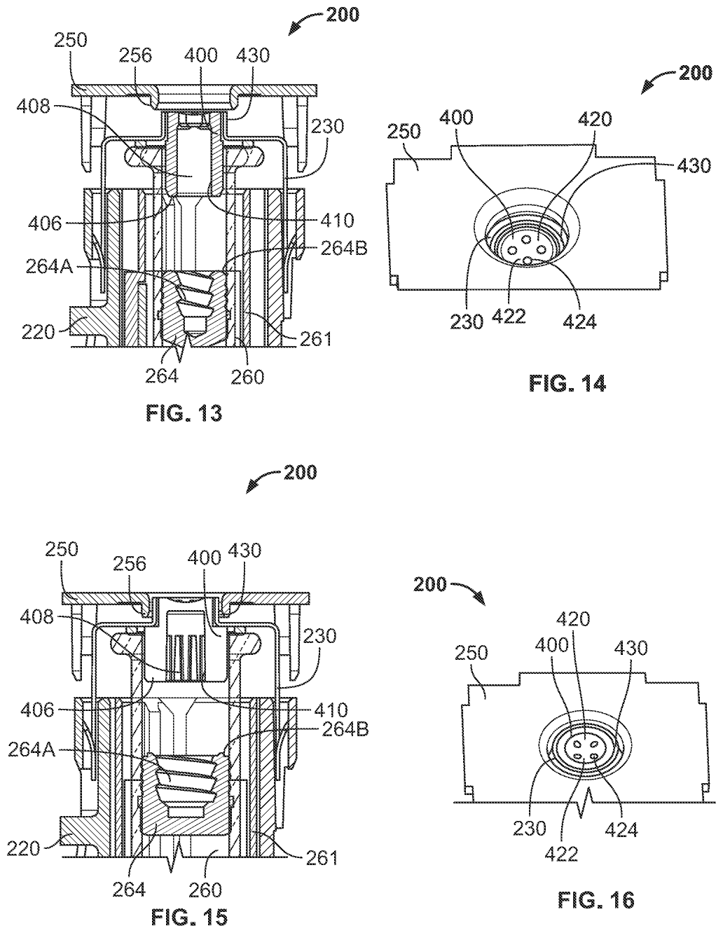

In the embodiments shown in FIGS. 9 and 10, the spacer 400 can have a cylindrical body with one or more portions having equal or varying diameters configured for various cassette 200 and syringe 260 configurations and sizes. For example, the proximal end 402 has an outer diameter sized so that an end surface 406 thereof can properly engage a trailing surface 264B (FIGS. 13 and 15) of the plunger-stopper 264, such that when the spacer 400 is pushed by the plunger rod 342, the spacer 400 drives the plunger-stopper 264 through the syringe 260 at a desired rate without undesirable slippage. In some versions, the proximal end 402 can be sized so that an outer surface 412 thereof can engage an interior surface of the barrel 261. For example, the proximal end 402 can be sized to engage the barrel 261 to resist movement by mass forces, such as gravity and inertia. Preferably, in these forms, the proximal end 402 can be sized to resist movement by mass forces, but have minimal or no excess friction beyond that required to resist movement by mass forces. Further, the outer surface 412 preferably orients the spacer 400 and radially fixes the spacer 400 in the barrel 261 by engaging the barrel 261 along a longitudinal length and/or at longitudinally spaced points thereof. In the illustrated form, the outer surface 412 of the proximal end 402 can include an array of grooves 414 that extend a longitudinal length of the proximal end 402 to provide a bypass for any air or other gas trapped between the spacer 400 and the plunger-stopper 264 when the spacer 400 is driven to the plunger-stopper 264. The grooves 414 can extend longitudinally along the proximal end 402 as shown, can have spiral configurations, or combinations thereof.

In the illustrated forms, the plunger-stopper 264 can have a cup-shaped configuration defining a rearwardly opening cavity 264A and an annular distal end surface 264B (FIGS. 13 and 15). As such, in one example, the spacer 400 can have a cavity 408 defining a hollow interior with an opening 410 defined in the end surface 406 giving the end surface 406 and at least a portion of the proximal end 402 an annular configuration. The annular end surface 406 of the proximal end 402 can be sized to engage the distal end surface 264B of the plunger-stopper 264, which advantageously avoids any issues that could arise between the relative sizes of the plunger rod 342 and the plunger-stopper 264, especially the cavity 264A thereof.

As described in more detail below, the distal end 404 of the spacer 400 couples to the cassette 200 to thereby secure the spacer 400 in position aligned with the plunger-stopper 264. For example, the distal end 404 can be configured to be press-fit into engagement with the sleeve 220, such as the lock cap 230 or other component thereof, to mount the spacer 400 to the cassette 200. Pursuant to this, an outer surface 416 of the distal end 404 can be sized to engage an opening of the sleeve 220. For example, the distal end 404 can be configured to engage an interior surface of the lock cap 230 or component to thereby resist movement by mass forces, such as gravity and inertia. Preferably, in these forms, the distal end 404 can be sized to resist movement by mass forces, but have minimal or no excess friction beyond that required to resist movement by mass forces. In the illustrated form, the outer surface 416 of the distal end 404 is provided by an array of radially extending ribs 418 that establish the outer diameter of the distal end 404, which minimizes a contact surface between the spacer 400 and the lock cap 203 or component. The ribs 418 can extend longitudinally along the distal end 404 as shown, or can have spiral configurations, or combinations thereof. The ribs 418 can also function to provide vents along the outer surface of the distal end 404. An end surface 420 of the distal end 404 is configured to be engaged by the plunger rod 342 and, as such, can have a configuration complementary to a leading surface of the plunger rod 342. For example, the end surface 420 can have a concave configuration as shown.

As shown in FIGS. 13 and 15, the cavity 408 of the spacer 400 can extend to an end wall 422 of the distal end 404 to reduce a weight and material cost associated with the spacer 400. Moreover, the end wall 422 can include one or more through openings 424 extending therethrough to allow air or gas trapped between the spacer 400 and the plunger-stopper 264 to escape therethrough when the spacer 400 is driven to the plunger-stopper 264.

In some embodiments, the proximal and distal ends 402, 404 can be separated by a neck 426 having a reduced outer diameter relative to the proximal and distal ends 402, 404. With this configuration, the neck 406 defines an annular space for reception of additional mounting structure, as discussed in more detail below.

Advantageously, the lock cap 230 described above can be modified to have the spacer 400 coupled thereto. In these forms, when the spacer 400 is coupled to the lock cap 230 and the lock cap 230 is secured to the sleeve 220, the spacer 400 is aligned with the barrel 261 and the plunger-stopper 264 disposed therein.

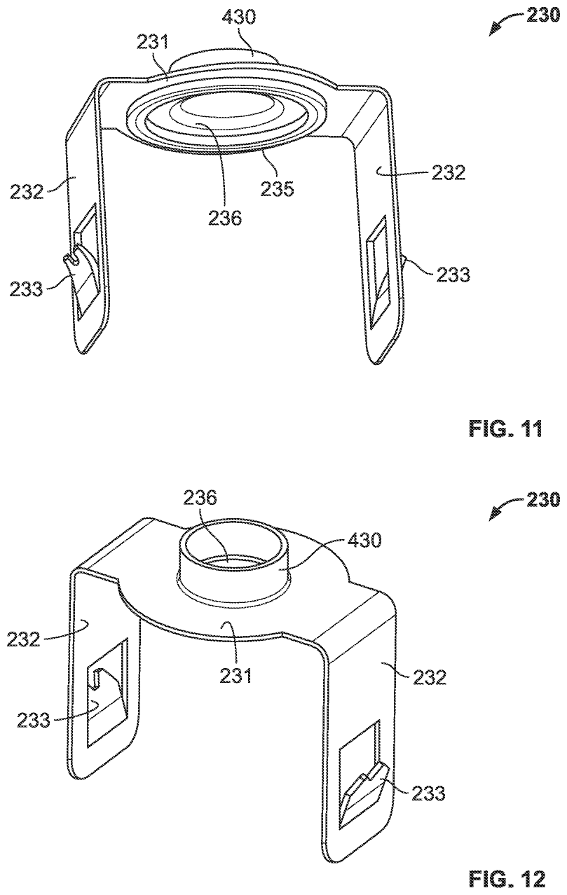

In a first form, as shown in FIGS. 11-16, the lock cap 230 can include an annular wall 430 that extends around the opening 236 and extends rearwardly from the body 231. The annular wall 430 defines a cylindrical cavity to receive the distal end 404 of the spacer 400 therein. As discussed above, the distal ends 404 of the various spacers can have a uniform configuration so that the same lock cap 230 can be utilized for spacers 400 having a variety of proximal ends 402. The inner diameter of the annular wall 430 and the outer diameter of the distal end 404 of the spacer 400 can advantageously be sized so that the lock cap 230 and spacer 400 engage one another in a press-fit arrangement. As shown in FIGS. 15 and 16, the distal end 404 can be sized so that the end surface 420 is at least partially co-planar with a top of the annular wall 420 when fully inserted therein.

In this form, the cover 250 can include an annular wall 256 that extends around the opening 254 forwardly towards the lock cap 230 to accommodate for the increased depth of the lock cap 230 provided by the annular wall 430. The annular walls 430, 256 are preferably sized so that the lock cap 230 and cover 250 tightly engage one another when they are mounted to the sleeve 220. Further, as shown in FIGS. 15 and 16, the annular walls 430, 256 can be sized so that the end surface 420 of the distal end 404 extends to an outer surface of the cover 250 to be generally, e.g., between 0 and 2 mm, coplanar therewith. Alternatively, the spacer 400 can be configured to engage the annular wall 256 of the cover 250 to couple the spacer 400 to the cassette 200.

With this configuration, during assembly, a user can select a spacer 400 having dimensions suitable for the particular plunger-stopper 264 and syringe 260 being used in the assembly. Thereafter, the distal end 404 of the spacer 400 can be press-fit into the opening of the annular wall 430 of the lock cap 230 and the lock cap 230 can be coupled to the sleeve 220. Finally, the cover 250 can be mounted to the sleeve 220 to complete the sleeve 220 assembly. After assembly, the injector 300 can push the spacer 400 out of engagement with the lock cap 230 during the extrusion process. Advantageously, this configuration provides a simplified assembly process and an extrusion process that creates no extra debris. In one example, the lock cap 230 can be made from metal and the spacer 400 can be made from plastic by any suitable process, such as injection molding.

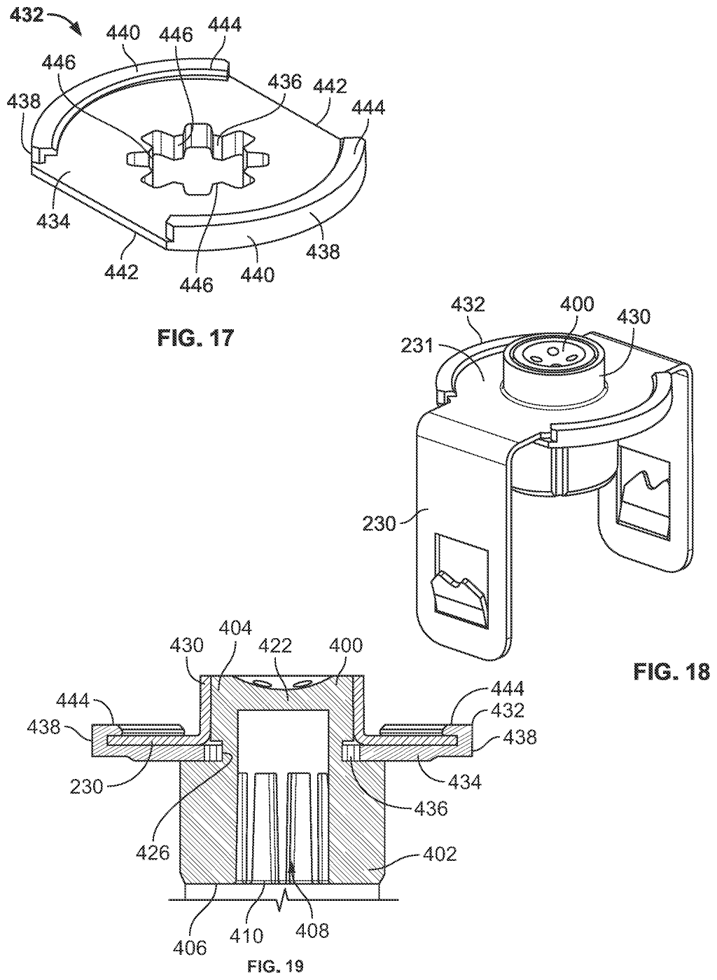

In a second, further form, shown in FIGS. 17-19, in addition to the annular walls 430, 256, the lock cap 230 can include a gasket 432 that couples to the body 231. The gasket 432 includes a main face 434 having a planar configuration with a central opening 436 extending therethrough and rims 438 extending from opposite curved edge portions 440 of the main face 434. The main face 434 has an annular configuration with straight edge portions 442 extending between the curved edge portions 440. With this configuration, the gasket 432 can couple to the body 231 of the lock cap 230 by lips 444 of the rims 438 extending over opposite edges of the body 231 and the legs 232 extending between the rims 438 and down over the straight edge portions 442. Of course, the perimeter of the gasket 432 can have any suitable form to couple to the body 231.

As shown in FIG. 19, in one example, a diameter of the central opening 436 can be sized to extend into the annular space defined by the neck 426 of the spacer 400, such that at least a portion of the gasket 432 extends between the proximal and distal ends 402, 404 of the spacer 400. The gasket 432 can be made from a deformable material, such as rubber or plastic, so that the spacer 400 can be pushed through the opening 436 by deforming the main face 434. In one configuration, the edge of the central opening 436 can define a plurality of teeth 446. With this configuration, the gasket 432 can provide a cushion between the lock cap 230 and sleeve 220 and absorb vibrations during transportation or accidental drops. The gasket 432 can also reduce the risk a glass-syringe breakage.

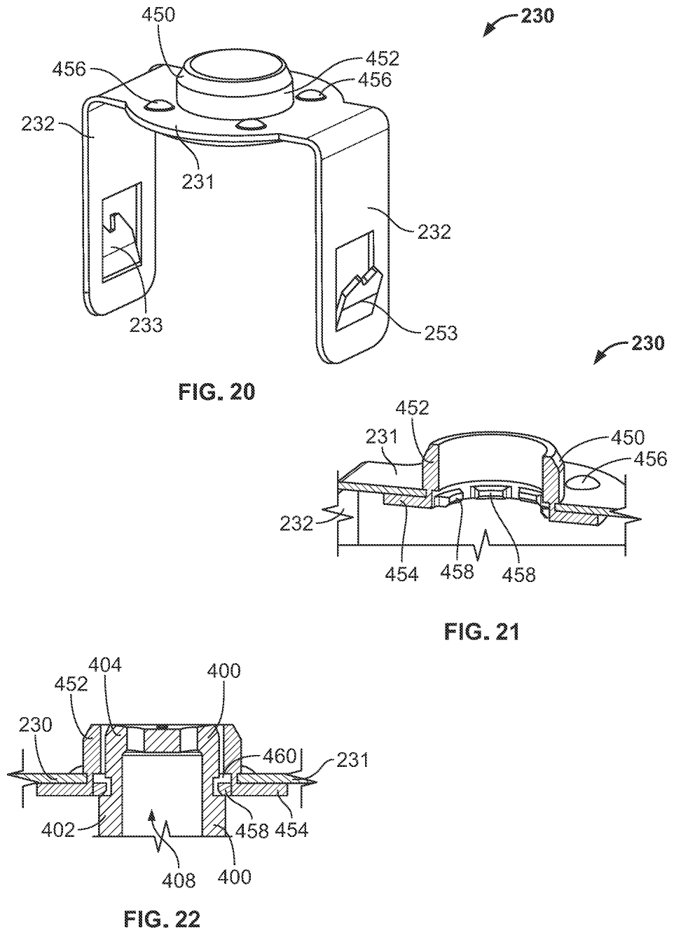

In a third form, shown in FIGS. 20-22, an overmolded member 450 including an annular upstanding wall 452 and a flange 454 that extends outwardly from a bottom edge of the wall 452 can be coupled to the body 231. In the illustrated form, the flange 454 extends along an interior surface of the body 231 and includes connection posts 456 that extend through openings in the body 231. As with the above forms, the interior diameter of the wall 452 can be sized to receive the distal end 404 of the spacer therein in a press-fit arrangement. If desired, in some examples, the wall 452 can further include inwardly extending teeth 458 that are configured to be inserted into the neck 426 of the spacer 400 and deformed during extrusion, as discussed above. The teeth 458 can be provided in an undercut 460 formed in the wall 452 as shown.

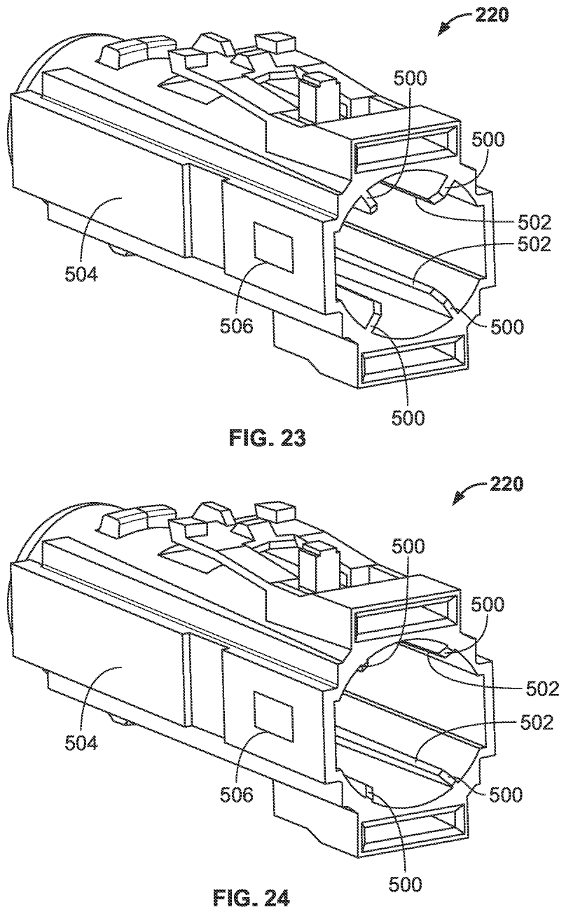

As shown in FIGS. 23 and 24, the sleeve 220 can also be modified to accommodate syringes 260 of varying sizes, while having uniform outer dimensions and a uniform configuration such that the same outer housing 210 and injector 300 can be utilized with the sleeves 220 and syringes 260. Pursuant to this, the sleeve 220 can include one or more internal walls or ribs 500 that define a mounting surface 502 for reception of the syringe 260. For example, the sleeve 220 can include an array of ribs 500, such as three, four as shown, five, or more, distributed radially about the interior of the sleeve 220 that cooperate to form a diameter suitable to support and position the syringe 260 within the sleeve 220. The radial length of the ribs 500 can be varied to accommodate syringes 260 having a variety of diameters. The ribs 500 support the syringe 260 without imparting any linear or rotational motion to the syringe 260. With this configuration, the sleeve 220 is only limited by dimensions of an outer wall or walls 504 with regard to the size of syringes 260 the system is able to accommodate. The sleeves 220 can also include an identifier 506 on an exterior thereof that allows users to easily identify and select a desired size for a particular syringe 260 during assembly. The sleeve 220 can be formed by any suitable process, such as injection molding. To create ribs 500 having a variety of dimensions, in one example, interchangeable cores can be utilized with a common exterior mold during an injection molding process.

It will be understood that the configurations described herein can be utilized with the sleeve 220 and the housing 210 to form a portion of the cassette 200. Further, the cassette 200, having the spacer 400 therein, can be inserted into the autoinjector 300 as described herein. As such, during a drug extrusion operation, the plunger rod 342 can be driven longitudinally through the autoinjector 300 to engage the spacer 400 and drive the spacer 400 through the barrel 261 to engage the plunger-stopper 264 and thereafter drive the spacer 400 and the plunger-stopper 264 through the barrel 261 to extrude a dose of a drug from the syringe 260.

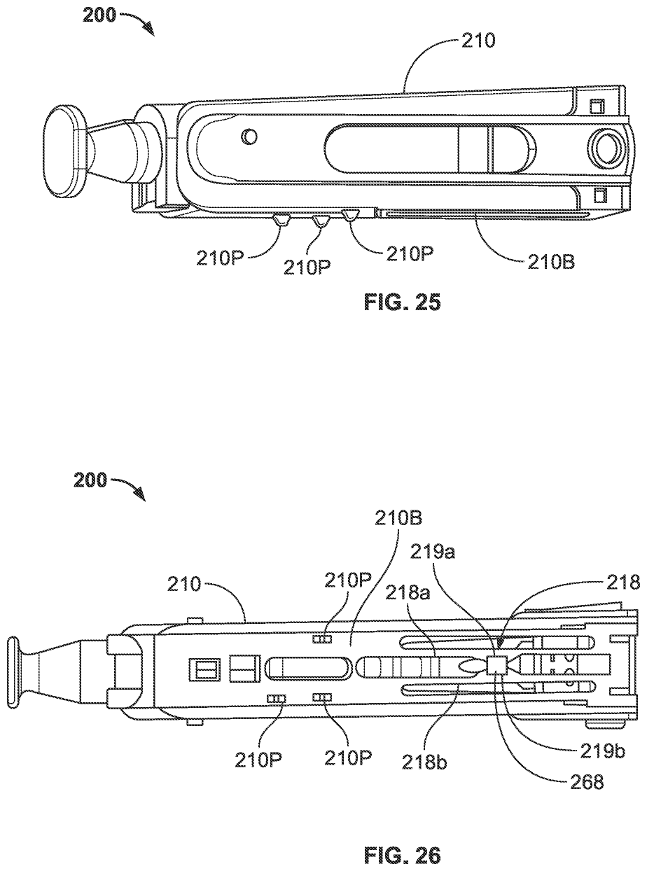

Referring to FIGS. 25 and 26, the outer housing 210 of the cassette 200 may comprise a cassette identification arrangement which provides information that identifies the cassette 200, e.g., information about the contents of the syringe 260 contained within the cassette 200 and/or other cassette/syringe characteristics. In one exemplary embodiment, the cassette identification arrangement may comprise one or more bumps or projections 210P provided on a bottom surface 210B of the outer housing 210 of the cassette 200. The projection(s) 210P may be sensed by or engage a detector (not shown) in the autoinjector 300 when the cassette 200 is inserted into the door 308 of the autoinjector 300 and the door 308 is closed. The detector 370 may be electrically coupled to a microprocessor (e.g. microprocessor 350 illustrated in FIG. 33) contained within the autoinjector 300, which enables the autoinjector 300 to read the cassette identification arrangement to thereby identify the cassette 200. In one exemplary embodiment, a predetermined number of projections 210P may be located on the bottom surface 210B of the outer housing 210 in predetermined locations, and the detector 370 may comprise a key pad of plural keys (not shown). Certain ones of the plural keys may be actuated by the cassette projections 210P when the cassette 200 is installed in the autoinjector 300, depending upon the location and number of the projections 210P. Each key actuated by one of the projections 210P may provide information that allows the autoinjector 300 to identify the cassette 200. In some embodiments, the cassette identification arrangement identifies the drug delivery profile of the pharmaceutical product provided in the cassette 200. Therefore, upon insertion and recognition of a valid cassette and the information provided by cassette identification arrangement, available preset drug extrusion speed ranges commensurate with the drug delivery profile of the pharmaceutical product provided in the cassette 200 may be automatically registered by the autoinjector 300. Available speed ranges are dependent upon the syringe fill volume and pharmaceutical product characteristics, such as viscosity. For example, but not limitation, if the cassette identification arrangement comprises plural projections 210P, one projection may indicate a 1 mL fill and two projections may indicate a 0.5 mL fill and additional projections may be provided to identify the pharmaceutical product and/or characteristics.

FIG. 26 also illustrates a latch mechanism 218 that may be provided on the bottom wall 210B of the outer housing 210 of the cassette 200. The latch mechanism 218 may include a pair of parallel extending, resilient locking arms 218a, 218b. The locking arms 218a and 218b may each define a locking detent slot 219a and 219b, respectively. The pin 268 of the inner sleeve 220 may engage the detent slots 219a, 219b of the latch mechanism 218 when the syringe 260 is in a home position with the injection needle 265 of the syringe 260 concealed in the cassette 260 in a needle concealed position, thereby locking of latching the inner sleeve 220 into place within the outer housing 210 of the cassette 200. During an injection cycle, the insertion drive 330 of the autoinjector 300 (FIG. 33) may spread the resilient locking arms 218a, 218b apart to unlatch or release the inner sleeve pin 268 from the detent slots 219a, 219b of the latch mechanism 218, thereby allowing the unlatched inner sleeve 220 containing the syringe 260 to be freely moved by the insertion drive 330, which pushes on the inner sleeve pin 268 to move the inner sleeve 220 relative to the outer housing 210 from the home position, where the injection needle 265 is in the needle concealed position, to an injection position, where the injection needle 265 is in a needle extended position that allows it to penetrate the skin at the injection site. At the end of the injection, cycle, the insertion drive 330 pulls the inner sleeve pin 268 back into the detent slots 219a, 219b, thereby returning the inner sleeve 220 (which contains the syringe 260) to the home position, where the injection needle 265 is in the needle concealed position.

Cassettes of similar structure and operation are described in greater detail in the following patent applications, each of which is incorporated herein by reference in its entirety: US Publ. Nos. 2009/0292246 and 20100022955; and PCT Publ. No. WO 2009/143255.

Referring again to FIGS. 2-4, the cover 250 attaches to a distal end of the outer housing 210 of the cassette 200 to close a distal end of the cassette 200. The cover 250 may be a generally planar member having a shape which matches that of the distal end 216 of the outer housing 210. The cover 250 may comprise two or more locking arms 253 that extend from an inner surface 251 of the cover 250 and lockingly engage corresponding receptacles 255 extending through the side walls 211 of the outer housing 210. In addition, any detent structure or other suitable locking arrangement (not shown) formed in, on, or through the outer housing 210, adjacent to the distal end 216 thereof may be used for attaching the cover 250. The cover 250 may further comprise an opening 254 which axially aligns with the opening 236 defined by the lock cap 230. The opening 254 in the cover 250, like the opening 236 of the lock cap 230, may be dimensioned to allow the plunger rod 342 actuated by the motorized extrusion drive 340 of the autoinjector 300 (FIG. 33), to pass through the cover 250 and engage and move the plunger-stopper 264 through the fluid chamber 262 of the syringe barrel 261 during the operation of the autoinjector 300.

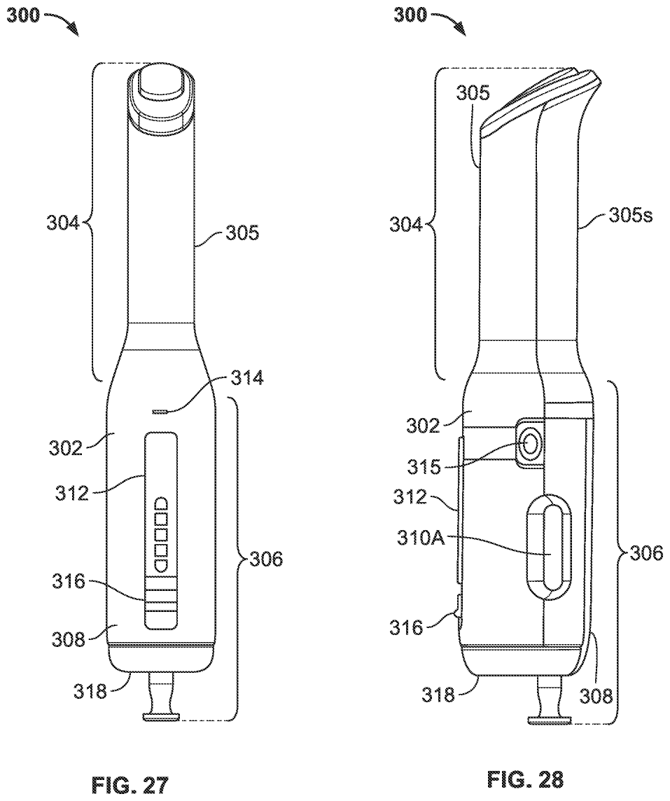

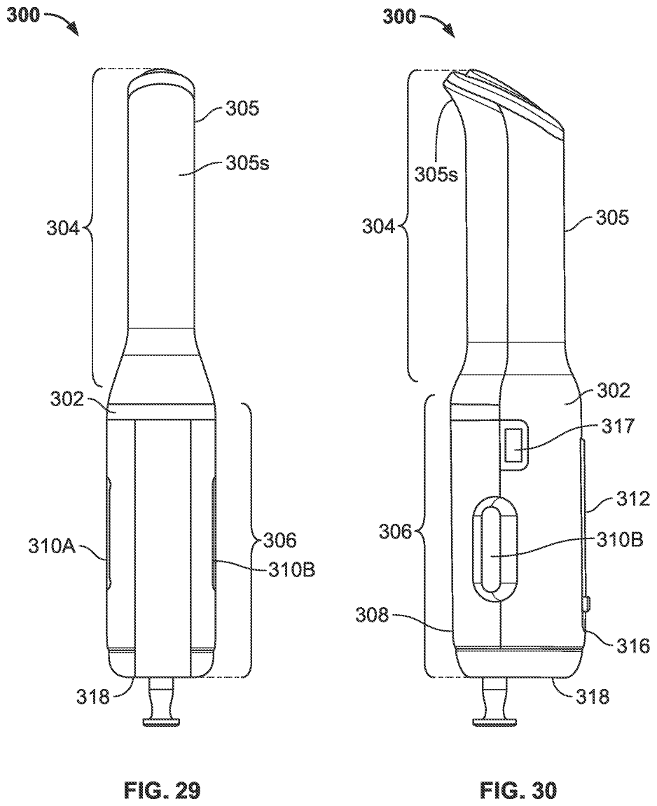

Referring now to FIGS. 27-32, the autoinjector 300 may comprise a casing 302 having a handle section 304 and a cassette receiving section 306 inline with the handle section 304. To aid patients with manual dexterity issues, the handle section 304 of the autoinjector casing 302 may define an ergonomically shaped handle 305 with a soft grip area 305S. The cassette receiving section 306 comprises the cassette door 308 (FIGS. 28 and 30) described earlier. The cassette door receives the cassette 200 in an open position (FIG. 1) and aligns the cassette 200 with insertion and extrusion drives, and other structures and components of the autoinjector 300 in a closed position. The cassette door 308 may include a “cassette” icon that indicates the insertion entry point for the cassette 200. The cassette receiving section 306 of the casing 302 may comprise windows 310A, 310B on opposing sides thereof that align with the windows 212 (FIG. 3) of the cassette 200 when the cassette door 308 is closed with the cassette 200 correctly installed therein. In one or more embodiments, the windows 310A, 310B may be double-layered. One or more lights (not shown) may be provided inside the casing 302 to evenly backlight illuminate the cassette windows 212 and the syringe 260 disposed within the inner sleeve 220 of the cassette 200, so that the user can observe the injection cycle through the windows 310A, 310B of the autoinjector 300, i.e., observe the initial and end positions of the plunger-stopper 264 of the syringe 260 during the syringe content (hereinafter “drug”) extrusion process, as well as syringe movements within the cassette 200.

Referring still to FIGS. 27, 28, 30, and 32, the autoinjector 300 may further comprise a user interface 312 and an audio speaker (not shown). The user interface 312 (best illustrated in FIG. 27) may be located in the cassette receiving section 306 of the casing 302, and provides various visual indicators. The audio speaker may be disposed inside the casing 302 and provides various audible indicators. The audio speaker may audibly communicate with the external environment via a speaker aperture 314 formed in the casing 302 in the cassette receiving section 306. The visual and audible indicators generated by the user interface 312 and the audio speaker can tell the user when the autoinjector 300 is ready for use, the progress of the injection process, injection completion, the occurrence of any errors, and other information. The autoinjector 300 may further comprise one or more of a settings/mute switch 315, a speed selector switch 316, a start button 307, and an eject button 317. The settings/mute switch 315 (FIG. 28) may be located in the cassette receiving section 306 of the casing 302. The mute switch 315 may be constructed and adapted allow the user to turn on and off all synthesized sounds, except error sounds, and to respond in real-time so that if the user begins the injection process and changes the mute switch to off, the sounds are immediately muted. The mute switch 315 may also be constructed and adapted to slide toward a “mute” icon to mute the audio speaker. A light indicator may be provided to confirm the “mute” state. The speed selector switch 316 (FIGS. 27 and 28) may be located in the cassette receiving section 306 of the casing 302. The speed selector switch 316 may be constructed and adapted to allow the user to select among a plurality of preset drug delivery (extrusion) speeds to accommodate personal patient preference. The speed selector switch 316 may comprise a three switch positions. Other embodiments of the speed selector switch may comprise two switch positions, or 4 or more switch positions. In still other embodiments, the speed selector switch may be of the infinitely variable type. In some embodiments, changing the position of the switch 316 prior to injection changes the speed of drug extrusion during injection while changing the position of the speed selector switch 316 during injection, does not change the speed of the injection in real time. The autoinjector 300 may also be provided with one or more demo cassettes to allow the user to experiment with different speeds of drug delivery. The start button 307 at a free end of the handle 305. The button 307 may include an indentation 3071 for optimizing thumb placement on the button 307. The button 307 may be made of a translucent material that allows a lighting effect to illuminate the button as signals. The eject button 317 (FIG. 30) may be located in the cassette receiving section 306 of the casing 302. The eject button 317 may include an indentation 3171 for optimizing finger placement on the button 317. In some embodiments, the eject button 317 may be controlled by the microprocessor (e.g. microprocessor 350 illustrated in FIG. 33) of the autoinjector 300, which may be programmed to eliminate accidental inputs during the injection process.

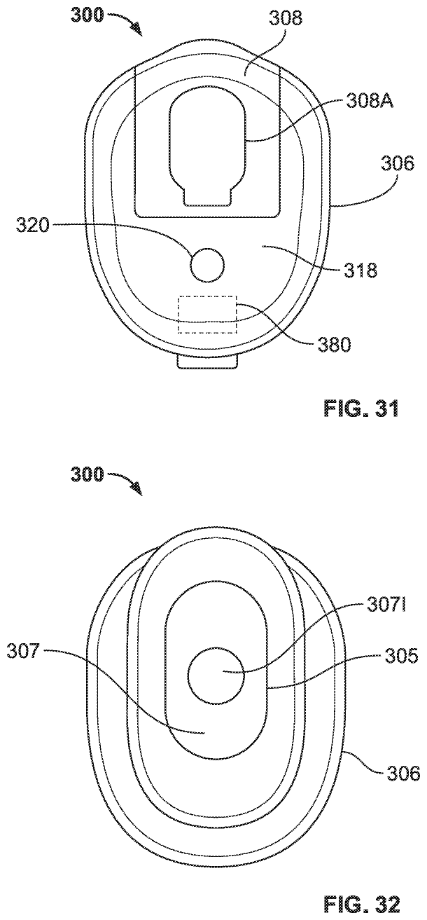

Referring again to FIG. 31, the cassette receiving section 306 of the casing 302 and the cassette door 308 may form a proximal end wall 318 of the autoinjector 300. The proximal end wall 318 may be configured as a broad, flat and stable base for easily positioning the autoinjector 300 on a support surface, after removal of the shield remover 240 or when the autoinjector 300 does not contain the cassette 240. The portion of the proximal end wall 318 formed by the cassette door 308 may include an aperture 308A that is sized and shaped to allow the shield remover 240 to be removed from the cassette 200 and withdrawn through the aperture 308A, when the cassette 200 is installed in the autoinjector 300. As soon as the shield remover 240 passes out through the aperture 308A, the tongues 245T of the expandable partial collar structure 245 expand or spread outwardly, thereby preventing the shield remover 240 and the needle shield 266 attached thereto from being re-inserted into the aperture 308A of the cassette door 308. The proximal end wall of the autoinjector 300 may further comprise a target light 320. The target light 320 may be constructed and adapted to turn on when the shield remover 240 is removed from the cassette 200 and withdrawn through the aperture 308A, thereby visually indicating that the shield remover 240 has been removed. Once turned on, the target light aids the user in visualizing and selecting an injection site.

FIG. 33 illustrates a sectional side view of the autoinjector apparatus 100 comprising the autoinjector 300 and the cassette 200 installed therein. The casing 302 of the autoinjector 300 may house a chassis 301 for receiving the cassette 200 that contains the syringe 260, a motorized insertion drive 330, a motorized extrusion drive 340, a microprocessor 350 (described earlier), a battery 360 for powering the drives 330, 340 and the microprocessor 350, and the skin sensor 380 (described earlier).

The microprocessor 350 may be programmed with certain instructions that executed by the microprocessor 350 enable it to control and monitor the various operations and functions of the autoinjector 300. For example, but not limitation, the microprocessor may be programmed with instructions for controlling the motorized insertion and extrusion drives 330, 340 such that it controls and monitors each step of the injection cycle and process flow, thereby automating needle insertion, drug extrusion, and needle retraction and ensuring accurate, consistent, and reliable operation of the autoinjector 300 and pharmaceutical product administration. The microprocessor may also be programmed with instructions for controlling the audible and visual feedbacks to the user. An automated power-on self-test checks the operation of the autoinjector 300 and remaining battery charge.

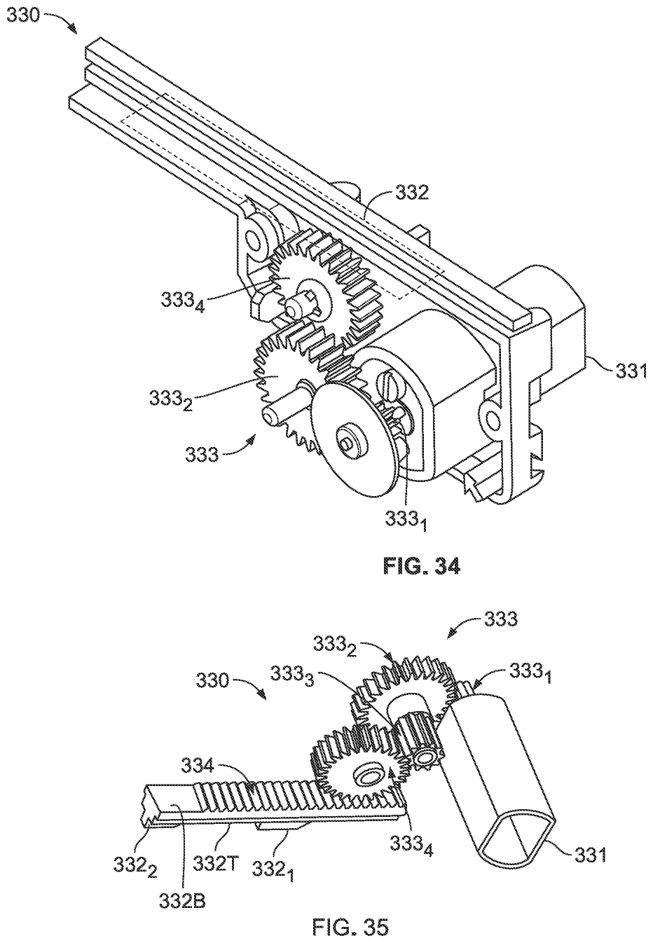

Referring again to FIG. 33, the motorized insertion drive 330 performs a needle insertion cycle and a needle retraction cycle. FIGS. 34 and 35 respectively illustrate a top down perspective side view and a bottom up perspective side view of an embodiment of the motorized insertion drive 330. The insertion drive 300 may comprise an insertion drive motor 331, a drive link or rack 332, and an insertion drive gear train 333 including a plurality of gears 3331, 3332, 3333, 3334, for transmitting the rotary motion of the insertion drive motor 331 to drive the rack 332. The rack 332 may include a top surface 332T and a bottom surface 332B. The top surface 332T of the rack 332 may include spaced-apart first and second protrusions, 3321 and 3322, respectively. The bottom surface 332B of the rack 332 may include rack teeth 334. The rack teeth 334 of the rack engage gear 3334 of the gear train 333. During a needle insertion cycle, the first protrusion 3321 of the rack 332 unlatches the inner sleeve pin 268 of the inner sleeve 220 of the cassette 200 from the latch 218 of the outer cassette housing 210 (FIG. 26) and then engages and then pushes the inner sleeve pin 268 to drive the inner sleeve 220 containing the syringe 260 forward within the outer housing of the cassette 200 from the home position to the needle extended position where the injection needle 265 of the syringe 260 extends out from the cassette 200 and is inserted into the skin at the injection site. During a needle retraction cycle, the second protrusion 3322 of the rack 332 engages and then pulls the inner sleeve pin 268 to drive the inner sleeve 220 containing the syringe 260 backward within the outer housing of the cassette 200 into the home position again, thereby withdrawing the injection needle 265 of the syringe 260 from the skin at the injection site and retracting it back into the cassette 200 (after drug extrusion) where the needle is shielded and locked within the cassette 200 for safe handling and disposal. The needle insertion positioning and timing are monitored and controlled by the microprocessor 350 of the autoinjector. If an error occurs, the error will be indicated on the user interface 312 (FIG. 27) along with audible alert from the speaker. The insertion drive 330 enables the autoinjector apparatus 100 to deliver the pharmaceutical product subcutaneously (SC) with a predetermined needle injection depth. This needle-depth parameter is accomplished when the insertion drive 330 moves the inner sleeve 220/syringe 260 forward to a mechanical hard stop within the outer housing 210 of the cassette 200. The mechanical hard stop limits the travel of the syringe 260 in the direction of the patient's skin, ensuring needle depth to the desired predetermined specification. Monitoring the movement of the motor 331 enables detection of incomplete needle insertion, which will trigger needle retraction and termination of the injection cycle, accompanied by audible and visual alerts.

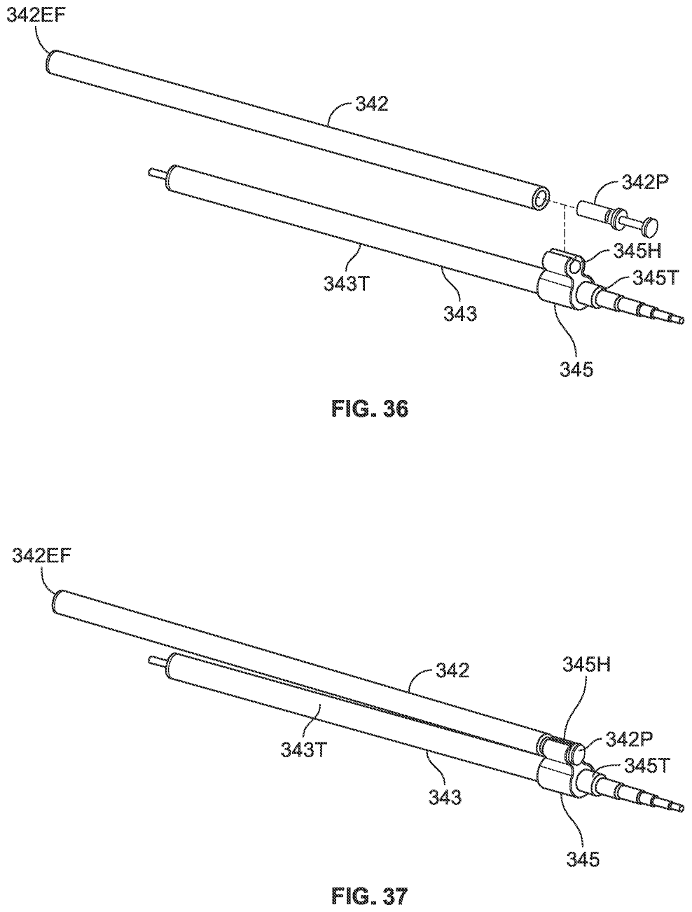

The motorized extrusion drive 340 illustrated in FIG. 33, performs the drug extrusion cycle where the pharmaceutical product is emptied from the syringe 260. FIGS. 36 and 37 are perspective side views illustrating an embodiment of the motorized extrusion drive 340. FIG. 36 illustrates an exploded perspective side view of an embodiment of a plunger rod/drive screw arrangement of the motorized extrusion drive 340. FIG. 37 illustrates an assembled perspective side view of the plunger rod/drive screw arrangement illustrated in FIG. 36. FIG. 38 illustrates a perspective view of an embodiment of a gear train of the motorized insertion drive 330. The extrusion drive 340 may comprise an extrusion drive motor 341, a plunger rod 342, a lead screw 343, and an extrusion drive gear train 344. The plunger rod 342 is driven by the extrusion drive motor 341 through the lead screw 343 and the extrusion drive gear train 344. As illustrated in FIGS. 36 and 37, the plunger rod 342 may include a pusher 342P and the lead screw 343 may include a nut 345. The nut 345 mechanically couples the plunger rod 342 to the lead screw 343. The nut 345 may include an internal screw thread 345T that threadedly engages an external screw thread 343T of the lead screw 343. The nut 35 may also include a holder 345H that fixedly holds the pusher 342P of the plunger rod 342. As illustrated in FIG. 38, the extrusion drive gear train 344 may include a plurality of gears 3441, 3442, 3443, 3444, 3445, 3446. The gears 3441 and 3446 of the extrusion drive gear train 344 are coupled to the extrusion drive motor 341 and the lead screw 343, respectively, thereby allowing the extrusion drive gear train 344 to transmit the rotary motion of the insertion drive motor 331 to drive the lead screw 343. As the lead screw 343 rotates, the nut 345 (which is threadedly engaged with the lead screw 343) moves forward or backward (depending upon the lead screw's direction of rotation) along the lead screw 343, which in turn, drives the plunger rod 342 forward and backward in the autoinjector 300. Forward movement of the plunger rod 342 causes an end face 342EF of the plunger rod 342 to enter the cassette 200 and subsequently the syringe barrel 261 of the syringe 260. The plunger rod 343 then engages the plunger-stopper 264 of the syringe 260 and pushes it to the end of the syringe barrel 261 in order to expel the predetermined dose of the pharmaceutical product from the syringe 260 during a drug extrusion cycle. The position of the components of extrusion drive 340, as well as time related to drug extrusion, may be monitored by the microprocessor 350. If an error occurs, the error can be indicated on the user interface 312 along with an audible alert. The microprocessor 350 may be capable of storing different factory-set drug delivery profiles (stroke, speed, acceleration). A plurality of unique drug delivery profiles may be associated with specific cassette configurations. The cassette identification arrangement on the outer housing 210 of the cassette 200 enable the autoinjector 300 to identify the proper drug delivery profile specific for the loaded pharmaceutical product. Upon insertion and recognition of a valid cassette 200, available preset drug extrusion speed ranges may be automatically registered by the autoinjector 300. Available speed ranges are dependent upon the syringe fill volume and pharmaceutical product characteristics, such as viscosity.

The user may select the desired drug extrusion speed (defined as the time to empty the pharmaceutical product of the syringe 260) from a plurality of different options for a particular pharmaceutical product using the speed selector switch 316. Upon initiation of the drug extrusion cycle, the stroke of the plunger rod 342 may be controlled and monitored to ensure the plunger-stopper 264 reaches the end of the syringe barrel 261, which ensures complete dose administration. If an error occurs during the extrusion process (e.g., failure of the plunger rod to achieve a complete stroke), the autoinjector 300 may immediately terminate drug extrusion, retract the needle back into the cassette 200, and provide audible and visual alerts.

The injection cycles may be indicated by both audible and visual signals. Lights on the autoinjector 300 may turn off in sequence from top to bottom during the injection cycle to indicate to the user the progress of the injection. Upon completion of the injection cycle, the autoinjector 300 retracts the syringe needle back into the disposable cassette 200, and then opens the cassette door 308 automatically, allowing removal of the cassette 200 by the user. The opening of the cassette door 308 may also be an indicator to the user that the injection cycle is complete.

In the event that an error occurs during the injection cycle, the autoinjector 300 may be equipped with various audible and visual signals to alert the user (operator or patient) to the error and to prompt appropriate actions.

The battery 360 illustrated in FIG. 33, may be a non-replaceable, non-rechargeable battery. In other forms, the battery 360 can be a replaceable battery and/or a rechargeable battery. The battery 360 should be capable of providing sufficient power for adequate shelf-life and service life to meet the drug delivery requirements. A power-on self-test is automatically performed upon waking the autoinjector 300 to ensure sufficient battery power is available for a successful injection cycle. The user interface 312 of the autoinjector 300 may provide visual and audible alerts if a problem occurs with the battery 360 before injection. The microprocessor 350 may be programmed to disable the autoinjector 300 at the end of the defined service life or if the battery 360 is not sufficiently charged for a successful injection cycle.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP lIb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(18)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vllI (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A cassette for a drug delivery device, the cassette comprising:
a sleeve having a proximal end and a distal end having an opening;
a syringe disposed in the sleeve, the syringe comprising a barrel having a distal opening coaxially aligned with the opening of the distal end of the sleeve;
a plunger-stopper slidably disposed within the barrel;
and
a spacer having a proximal end and a distal end, the distal end configured to be inserted into the opening to couple the spacer to the sleeve, the distal end of the spacer adapted to be engaged by a plunger rod of a drive mechanism to uncouple the spacer from the sleeve and slide the spacer within the barrel to engage the plunger-stopper with the proximal end thereof.

2. The cassette of claim 1, wherein the sleeve further comprises (a) an annular wall extending around the opening, the distal end of the spacer configured to engage an interior surface of the annular wall, and/or (b) a lock cap configured to secure the syringe in the sleeve.

3. (canceled)

4. The cassette of claim 2, wherein the sleeve further comprises the lock cap configured to secure the syringe in the sleeve, and wherein the opening of the sleeve is defined by a portion of the lock cap extending over the distal opening of the barrel, such that the spacer is configured to couple to the lock cap.

5. The cassette of claim 4, wherein the portion of the lock cap comprises a generally planar body, and the annular wall is integral with the body.

6. The cassette of claim 5, wherein the lock cap further comprises a gasket configured to couple to the body with a main face of the gasket extending along an interior surface of the body, the main face defining an opening configured to coaxially align with the opening of the body such that the distal end of the spacer is configured to be inserted through the opening of the gasket.

7. The cassette of claim 6, wherein the spacer further comprises a neck portion disposed between and having a reduced diameter relative to the proximal and distal ends to define a space therebetween, and the opening of the gasket has a diameter sized so that portions of the main face extend into the space between the proximal and distal ends of the spacer.

8. The cassette of claim 7, wherein the gasket includes teeth arrayed about and extending radially into the opening defined in the main face, the teeth at least partially extending into the space between the proximal and distal ends of the spacer.

9. The cassette of claim 6, wherein the gasket comprises one or more rims extending away from the main face, the rims including lips configured to engage the body to couple the gasket thereto.

10. The cassette of claim 4, further comprising a cover configured to couple adjacent to the distal end of the sleeve with the lock cap disposed proximal of the cover, the cover including an opening extending therethrough and an annular wall extending around the opening and extending in a proximal direction, such that the annular wall of the cover extends around the annular wall of the lock cap.

11. The cassette of claim 4, wherein
(a) the lock cap comprises a tubular member including the annular wall and a generally planar body having an annular configuration, the tubular member coupled to the body with the annular wall extending through the body, and the tubular member optionally including a flange extending along the body, the body and tubular member overmolded together with connection posts of the tubular member extending from the flange through openings in the body, and/or
(b) the spacer further comprises a neck portion disposed between and having a reduced diameter relative to the proximal and distal ends to define a space therebetween, and the tubular member includes one or more projections that extend radially inward from the annular wall, the projections sized to at least partially extend into the space between the proximal and distal ends of the spacer.

12-13. (canceled)

14. The cassette of claim 1, wherein the proximal end of the spacer has (a) a diameter approximately equal to a diameter of the plunger-stopper and/or (b) one or more grooves extending along an outer surface thereof.

15. (canceled)

16. The cassette of claim 1, wherein the distal end of the spacer (al includes a plurality of ribs extending radially outwardly therefrom, the plurality of ribs providing an outer diameter from the distal end to frictionally engage the lock cap, and/or (b) is configured to be coplanar with a distal end surface of the sleeve with the spacer coupled thereto.

17. The cassette of claim 1, wherein the spacer has a cup-shaped configuration with a distal end wall and a cavity having an opening extending through the proximal end, wherein the distal end wall optionally defines one or more vent openings extending therethrough.

18-19. (canceled)

20. The cassette of claim 1, further comprising (a) an outer housing configured to movably receive the sleeve and syringe therein, and/or (b) a therapeutic product in the syringe.

21-22. (canceled)

23. A method for preparing a cassette for an autoinjector, the method comprising:
disposing a plunger-stopper within a barrel of a syringe;
disposing the syringe within a sleeve;
inserting a distal end of a spacer into an opening of the sleeve to couple the spacer thereto, the opening being aligned with a distal opening of the barrel of the syringe to coaxially align the spacer with the barrel of the syringe.

24. The method of claim 23, wherein inserting the distal end of the spacer into the opening of the sleeve comprises inserting the distal end of the spacer into an opening of a lock cap to couple the spacer thereto; and further comprising coupling the lock cap to a distal end of the sleeve such that the spacer is coaxially aligned with the distal opening of the barrel of the syringe.

25. The method of claim 24, wherein inserting the distal end of the spacer into the opening the lock cap further comprises inserting the distal end into a cavity defined by an annular wall of the lock cap, the distal end configured to frictionally engage an interior surface of the annular wall, wherein inserting the distal end of the spacer into the cavity defined by the annular wall further optionally comprises inserting the distal end of the spacer through an opening in a gasket or member coupled to a body of the lock cap such that a portion of the gasket or member extends into a space between the distal end of the spacer and a proximal end of the spacer.

26. (canceled)

27. The method of claim 23, further comprising:
(a) selecting the spacer based on a size of the plunger-stopper and a size of a proximal end of the spacer,
(b) selecting the sleeve based on a size of an interior bore defined by one or more interior walls of the sleeve and a diameter of the barrel of the syringe, and/or
(c) filling the syringe with a therapeutic product.

28-29. (canceled)

30. A method of assembling a cassette for a drug delivery device, the method comprising:
selecting a syringe having a barrel with an outer diameter;
selecting a sleeve from first and second sleeves, the first and second sleeves having a common outer configuration, common outer dimensions, and internal bores defined by one or more walls having different diameters, the selection of the sleeve comprising selection of one of the first and second sleeves having an internal bore sized to support the barrel of the syringe;
inserting the syringe into the sleeve; and
inserting the syringe and sleeve into a housing, the housing configured to couple to the common outer configuration of the first and second sleeves.

31. The method of claim 30, wherein the internal bores of the first and second sleeves are defined by an array of radial ribs extending within an interior of the sleeve.
